(19) United States
(12) Patent Application Publication
(10) Pub. No.: US 2023/0022031 A1
(43) Pub. Date: Jan. 26, 2023

(54) POWER PORT CONNECTOR FOR MEDICAL DEVICE

(71) Applicant: GYRUS ACMI, INC. D/B/A OLYMPUS SURGICAL TECHNOLOGIES AMERICA, Westborough, MA (US)

(72) Inventor: Kester Julian Batchelor, Mound, MN (US)

(21) Appl. No.: 17/758,503

(22) PCT Filed: Jan. 5, 2021
(86) PCT No.: PCT/US2021/012163

§ 371 Date: Jul. 7, 2022

Related U.S. Application Data
(60) Provisional application No. 62/957,934, filed on Jan. 7, 2020.

(51) Int. Cl. A61B 17/34; A61B 90/00

(52) U.S. Cl. CPC A61B 17/3421; A61B 90/361

(57) ABSTRACT

A medical device can include a surgical device (102) that can include an elongated shaft (118) configured to be guided via an access stabilizer (1224). The device can include a housing mechanically coupled to the shaft. The device can include an electrical port (122) at least partially around the shaft, the shaft extending through and able to longitudinally translate through an opening in the electrical port. The device can include one or more electrical interconnects (120) configured to receive an electrical signal from or provide the electrical signal to the electrical port.

### CROSS-REFERENCE TO RELATED APPLICATION

This application claims priority to U.S. Provisional Application Ser. No. 62/957,934 filed Jan. 7, 2020, the contents of which are incorporated herein by reference in their entirety.

### FIELD

These teachings relate to a medical device, and more particularly to providing electrical power to an elongated medical device.

### BACKGROUND

Endoscopic surgery can include using an endoscope to check for problems in an abdomen, such as via a body orifice or via a small incision that can be made through the skin of a patient. The endoscope can be inserted through the orifice or incision. A camera of the endoscope can relay image data for a surgeon to view. The images can help medical personnel identify trauma, abnormalities, or other conditions of a reproductive system, nostril, joint, airway, throat, larynx, trachea, or other feature in or around an opening in a patient. Illustrative examples of conditions that can be detected using an endoscope include tumors, blockages, bleeding, infections, fibroids, cysts, endometriosis, prolapse, ectopic pregnancy, or the like. Sometimes, an endoscope can include a cutting or grasping mechanism. Such endoscopes can be used to remove an ectopic pregnancy, perform a hysterectomy, perform a tubal ligation, or treat incontinence.

An endoscope can include a thin, elongated tube that includes a housing at a proximal end and a light, camera, cutting, grasping, other device, or a combination thereof at a distal end. In an endoscopy a patient can be anesthetized using a local or general anesthetic. The small incision can be made. A gas can optionally be provided internal to the patient to expand a target internal area and make it easier to see and move within the area. Another incision can be made for the surgeon to insert another endoscope, or a device like an endoscope to perform surgery. The surgery can be performed, the gas and endoscope(s) can be removed, and the incisions can be closed.

Operations with a corded endoscope in which the cord extends out a distal end of the endoscope can frustrate the surgeon. The cord can become tangled and reduce the free range of motion using the device. Operations with a cordless device provide the free range of motion but can have other problems. One such problem is interrupting the surgery to switch out a depleted battery. In such battery switching a seal between a cannula and the endoscope can be lost. Another problem with the battery powered device is weight. The weight of the device can fatigue the surgeon, especially over a surgery that lasts a significant amount of time e.g., a half hour, one hour, longer, or some time therebetween). Such fatigue can create secondary issues.

### SUMMARY

These teachings overcome one or more of the problems with prior devices discussed in the Background.

These teachings provide a surgical device comprising an elongated shaft configured to be guided via an access stabilizer, a housing mechanically coupled to the shaft, an electrical port at least partially around the shaft, the shaft extending through and able to longitudinally translate through an opening in the electrical port, and one or more electrical interconnects configured to receive an electrical signal from or provide the electrical signal to the electrical port. The electrical port can be configured to mate with a complementary electrical port mechanically coupled to or integrally formed with the access stabilizer.

The electrical port can be configured to break an electrical and mechanical contact with the complementary electrical port during removal of the shaft from the opening. The electrical signal can include electrical power from a power port of the electrical port. The electrical signal can include surgical device identification data to a data port of the electrical port. The electrical port can include a male or female electrical connection feature electrically coupled to the one or more electrical interconnects and configured to mate with a corresponding female or male connection feature of the complementary electrical port.

The electrical port can be keyed to the complementary electrical port. The keying can include at least one of a magnet or a contoured surface configured to align the electrical port with the complementary electrical port. The surgical device can further include a collapsible or rotatable shroud around the shaft, surrounding the electrical interconnects, and mechanically coupled to the housing. The shroud can be mechanically coupled to the electrical port. The surgical device can further include a retractable power cord between the electrical port and the housing that accommodates the longitudinal translation of the shaft through the electrical port. The one or more electrical interconnects can further pass through the hole in the electrical port and provide the electrical signal to a distal device on the shaft.

A surgical device can include an access stabilizer configured to guide an elongated shaft of an elongated surgical device, a complementary electrical port configured to attach to the access stabilizer or integrally formed with the access stabilizer, the complementary electrical port including an opening therethrough, and an electrical cord coupled to the electrical port to provide an electrical signal from a signal generator to the complementary electrical port. The surgical device can further include a male or female mechanical connection feature on the complementary electrical port. The surgical device can further include a female or male mechanical connection feature situated about the access stabilizer and configured to mate with the male or female mechanical connection feature of the complementary electrical port. The complementary electrical port can be integrally formed with the access stabilizer. The opening can be configured to allow the shaft of the elongated surgical device to extend and translate longitudinally therethrough.

The complementary electrical port can be configured to electrically connect to an electrical port of the elongated surgical device at a distal portion thereof. The complementary electrical port can be keyed to the electrical port. The keying can include one or more of a magnet or contoured surface to orient the complementary electrical port. The complementary electrical port can be configured to break an electrical and mechanical contact with the electrical port with removal of the shaft from the opening. The complementary electrical port can include male or female electrical connection features configured to electrically connect to corresponding female or male electrical connection features of the electrical port.

The complementary electrical port can include one or more of a power port or data port. The opening in the complementary electrical port can be concentric with an opening in the access stabilizer.

A surgical system can include a first surgical device and a second surgical device. The first surgical device can include an access stabilizer, a complementary electrical port configured to attach to the access stabilizer or integrally formed with the access stabilizer, and an electrical cord coupled to the electrical port to provide an electrical signal to or receive the electrical signal from the complementary electrical port. The second surgical device can include an elongated shaft configured to be guided via the access stabilizer, a housing mechanically coupled to the shaft, a second electrical port at least partially around the shaft, the shaft extending through and able to longitudinally translate through complementary openings in the complementary and second electrical ports, and one or more electrical interconnects configured to receive the electrical signal from or provide electrical signal to the complementary electrical port through the second electrical port.

The complementary electrical port can be configured to electrically and mechanically mate with the second electrical port. The complementary and second electrical ports can be configured to break the electrical and mechanical mating during removal of the shaft from the openings in the complementary and second electrical ports. The electrical signal can include electrical power from the complementary electrical port.

The electrical signal can include surgical device identification data. The complementary electrical port can include male or female electrical connection features configured to mate with corresponding female or male connection features of the second electrical port. The complementary electrical port can include a keying element complementary to a corresponding keying element of the second electrical port. The keying element can include at least one of a magnet or a contoured surface configured to align the complementary electrical port with the second electrical port.

The second surgical device can further include a collapsible shroud or rotatable around the shaft and surrounding the electrical interconnects. The shroud can be mechanically coupled to the second electrical port. The first surgical device can further include a male or female mechanical connection feature on the complementary electrical port, and a female or male mechanical connection feature about the access stabilizer and configured to mate with the male or female mechanical connection feature of the complementary electrical port. The complementary electrical port can be integrally formed with the access stabilizer.

A method of operating a surgical system can include situating an elongated shaft of a first surgical device through an access stabilizer of a second surgical device, and electrically and mechanically coupling a complementary electrical port of the first surgical device with a second electrical port of the second surgical device. The method can further include removing the shaft from the access stabilizer to break the electrical and mechanical coupling between the complementary and second electrical ports. The method can further include, before situating the elongated shaft through the access stabilizer, situating the second surgical device at partially in an incision or natural orifice.

The method can further include, before removing the shaft from the access stabilizer, moving the first surgical device to translate the shaft through openings in the complementary and second electrical ports. Moving the first surgical device can cause a shroud around the shaft to collapse or elongate. Electrically of mechanically coupling the complementary and second electrical ports includes mating complementary keying elements of the complementary and second electrical ports.

Mating complementary keying elements can include one or more of respective contoured surfaces or respective magnets. The electrical coupling can provide an electrical path for an electrical signal to be provided to the complementary electrical port from the second electrical port or to the second electrical port from the complementary electrical port. The electrical signal includes one or more of electrical power or data indicating a device identification of the first surgical device.

### BRIEF DESCRIPTION OF THE DRAWINGS

FIG. 1 illustrates, by way of example, a diagram of an embodiment of a surgical system.

FIG. 2 illustrates, by way of example, a diagram of an embodiment of the surgical device with a shaft removed from the opening in an electrical port.

FIG. 3 illustrates, by way of example, a diagram of an embodiment of an electrical port.

FIG. 4 illustrates, by way of example, a diagram of an embodiment of another electrical port.

FIG. 5 illustrates, by way of example, perspective view diagram of a portion of an embodiment of a surgical device.

FIG. 6 illustrates, by way of example, a diagram of an embodiment of a medical system.

FIG. 7 illustrates, by way of example, a diagram of an embodiment of an electrical port.

FIGS. 8, 9, and 10 illustrate, by way of example, perspective view diagrams of respective embodiments of the electrical port.

FIG. 11 illustrates, by way of example, a diagram of an embodiment of the surgical device in the form of a cannula.

FIG. 12 illustrates, by way of example, a diagram of an embodiment of the electrical port attached to the surgical device.

FIG. 13 illustrates, by way of example, a cross-section diagram of an embodiment of an electrical and mechanical mating between electrical ports.

FIG. 14 illustrates, by way of example, a diagram of an embodiment of another embodiment of the electrical port.

FIG. 15 illustrates, by way of example, a diagram of an embodiment of multiple electrical ports electrically coupled in parallel to a signal generator.

FIG. 16 illustrates, by way of example, a diagram of an embodiment of multiple electrical ports electrically coupled in series to the signal generator.

FIG. 17 illustrates, by way of example, a diagram of an embodiment of the electrical port that includes multiple proper orientations with the electrical port.

FIG. 18 illustrates, by way of example, a diagram of another embodiment of the electrical port.

FIG. 19 illustrates, by way of example, a diagram of yet another embodiment of the electrical port.

FIG. 20 illustrates, by way of example, a diagram of yet another embodiment of the electrical port.

FIG. 21 illustrates, by way of example, a perspective view diagram of an embodiment of an electrical port situated more proximal than at least a portion of an access stabilizer.

FIG. 22 illustrates, by way of example, a diagram of an embodiment of a surgical device that includes an electrical port configured to mate with the electrical port or the electrical port.

FIGS. 23-25 illustrate, by way of example, respective embodiments of electrical interconnect management devices.

FIG. 26 illustrates, by way of example, a diagram of an embodiment of the electrical interconnect not wound around the shaft.

FIGS. 27, 28, and 29 illustrate, by way of example, perspective view diagrams of steps for electrically and mechanically coupling and decoupling the electrical ports.

### DETAILED DESCRIPTION

These teachings provide a medical device or a system of medical devices. The medical device can include an elongated or other surgical device such as comprising an elongated shaft that can be configured to be guided such as via an access stabilizer. The access stabilizer can include a device that facilitates ingress into a cavity. The surgical device can include a housing, such as can be mechanically coupled to the shaft. The housing can be sized, shaped, or otherwise configured to be gripped by medical personnel, such as to facilitate motion of the surgical device into and out of an opening in a patient. The surgical device can include an electrical port, such as at least partially around the shaft, the shaft extending through and able to longitudinally translate through an opening in the electrical port. The surgical device can include one or more electrical interconnects configured to receive an electrical signal from or provide the electrical signal to the electrical port.

The electrical port can be coupled (e.g., electrically, mechanically, or both) with a complementary electrical port of an access stabilizer, such as can be mechanically coupled to the access stabilizer. The electrical port can be configured to break an electrical and mechanical contact with the complementary electrical port, such as upon removal of the shaft from the opening. A user can manipulate the shaft out of the opening and cause the electrical and mechanical contact with the complementary electrical port to be broken.

The electrical port can include a data port, such as through which identification data or other data can be provided to the complementary electrical port. The electrical port can include a power port such as through which electrical power can be received from the complementary electrical port. The identification data can be used, for example, to identify the type of surgical device or type of power to be provided, such that the appropriate electrical power can be provided to the surgical device via the power port of the electrical port. The electrical power can be from a signal generator and provided, by electrical interconnects, to a distal device on the shaft.

The electrical port can include a male or female electrical connection feature that can be electrically coupled to the one or more electrical interconnects of the surgical device. The electrical port can be configured to mate with a corresponding female or male connection feature of the complementary electrical port. Electrical power or data can be provided or received through the electrically conductive connection provided by mating between the one or more electrical interconnects and the mated electrical connection features.

The electrical port can be keyed to the complementary electrical port. The key can include at least one of a magnet or a contoured surface configured to align the electrical port with the complementary electrical port.

The surgical device can include a collapsible or rotatable shroud around the shaft and surrounding the electrical interconnects. The shroud, whether rotatable or collapsible, can be mechanically coupled to the electrical port (such as to not Obstruct the opening in the electrical port). The surgical device can include a spooling or other electrical interconnect management device, such as to help keep the electrical interconnects taut between the electrical port and the housing. The electrical interconnect management device can accommodate longitudinal translation of the shaft through the opening. The electrical interconnect management device can provide tension such as to help decouple the electrical port and the complementary electrical port.

In an example, the access stabilizer can be configured to guide the elongated shaft of the elongated surgical device. This access stabilizer can include the complementary electrical port. The complementary electrical port can be configured to attach to the access stabilizer or can be integrally formed with the access stabilizer. The complementary electrical port can include an opening therethrough such as concentric or overlapping with a lumen of the access stabilizer. This surgical device can include an electrical cord coupled to the complementary electrical port to provide an electrical signal to the complementary electrical port. The electrical signal can be from circuitry of the surgical device. The electrical signal can include data identifying the surgical device. The electrical signal can be provided to a signal generator, such as to indicate to the signal generator one or more parameters of an electrical signal to be provided to the surgical device. The electrical signal can be from the signal generator and provide electrical power to the surgical device, via the complementary electrical, the electrical port, and electrical interconnects electrically coupling the electrical port to the surgical device. The electrical signal can be provided to a distal device on the shaft of the surgical device.

The complementary electrical port can include a male or female mechanical connection feature on the complementary electrical port. The access stabilizer can include a female or male mechanical connection feature. The female or male connection feature can be configured to mate with the male or female mechanical connection feature of the complementary electrical port.

The opening in the complementary electrical port can be configured to allow the shaft of the elongated surgical device to extend and translate longitudinally therethrough. The electrical port can be configured to electrically connect to a complementary electrical port of the elongated surgical device, such as at a distal portion thereof. The electrical port can be keyed to the complementary electrical port. The keying, as previously discussed, can include one or more of a magnet or contoured surface, such as to help orient the complementary electrical port to the electrical port of the elongated surgical device.

The electrical port can be configured to break an electrical and mechanical contact with the complementary electrical port such as upon removal of the shaft from the opening. The complementary electrical port can include a female or male electrical connection feature configured to electrically connect to a corresponding male or female electrical connection feature of the electrical port. The complementary electrical port can include one or more of a power port or data port.

A surgical system can include the elongated surgical device and an access stabilizer (“other surgical device”). A method of operating the surgical system can include situating an elongated shaft of a first surgical device (the elongated surgical device) through an access stabilizer of a second surgical device (the other surgical device). The method can further include electrically and mechanically coupling a first electrical port of the first surgical device with a second electrical port of the second surgical device. The method can further include removing the shaft from the access stabilizer to break the electrical and mechanical coupling between the first and second electrical ports.

The method can further include, before situating the elongated shaft through the access stabilizer, situating the second surgical device at least partially in an incision or natural orifice. The method can further include, before removing the shaft from the access stabilizer, moving the first surgical device to translate the shaft through concentric openings in the first and second electrical ports. Moving the first surgical device can cause a shroud around the shaft to collapse.

Electrically and mechanically coupling the first and second electrical ports can include mating complementary keys or keyed features of the first and second electrical ports. The complementary keys can include one or more of respective contoured surfaces or respective magnets. The electrical coupling can provide an electrical path for an electrical signal to be provided to the first electrical port from the second electrical port or to the second electrical port from the first electrical port. The electrical signal can include one or more of electrical power or data such as indicating a device identification of the first surgical device or providing information about its type or about one or more of its operating parameters.

Reference will now be made to the FIGS. to describe embodiments and further details of the embodiments,

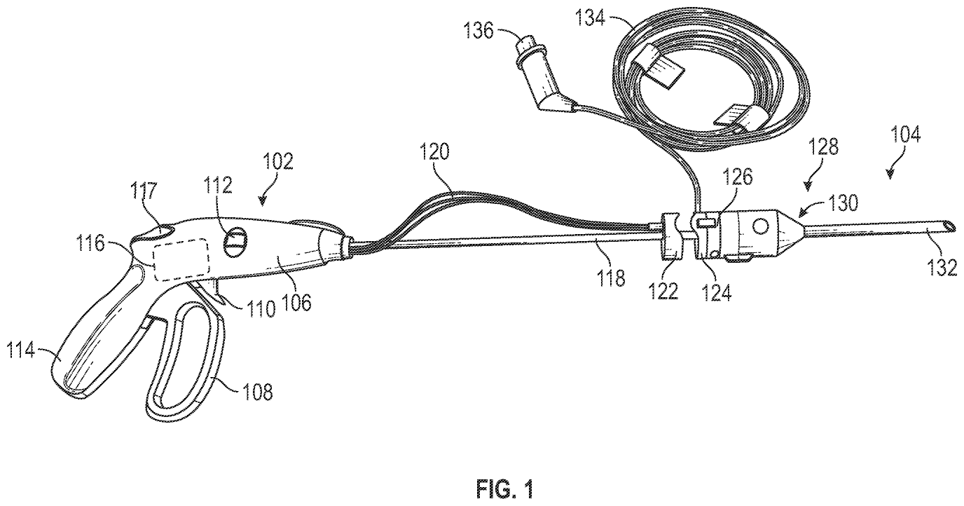

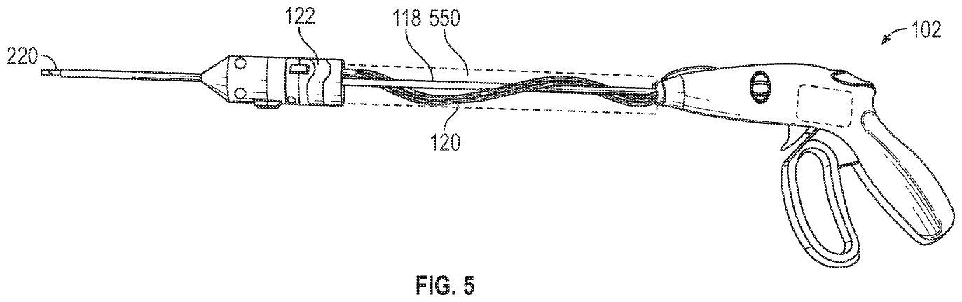

FIG. 1 illustrates, by way of example, a diagram of an embodiment of a surgical system 100. The surgical system 100 as illustrated can include a first surgical device 102 and a second surgical device 104. The first surgical device 102 can include a housing 106, one or more optional triggers 108, 110, a rotation wheel 112, a housing 114, optional circuitry 116, an elongated shaft 118, electrical interconnects 120, and an electrical port 122. The first surgical device 102, sometimes called the elongated surgical device, can optionally include a rotatable or collapsible shroud. For more details on an example of the shroud, see FIG. 5.

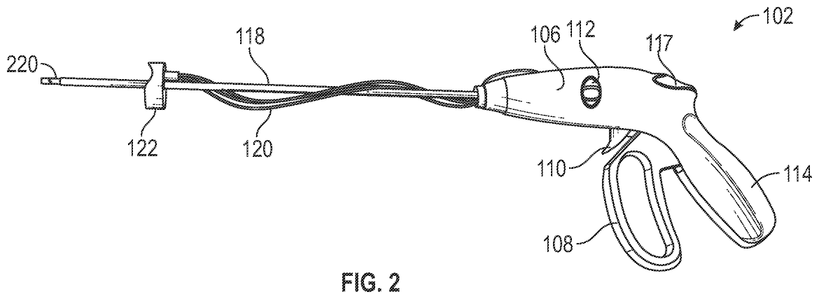

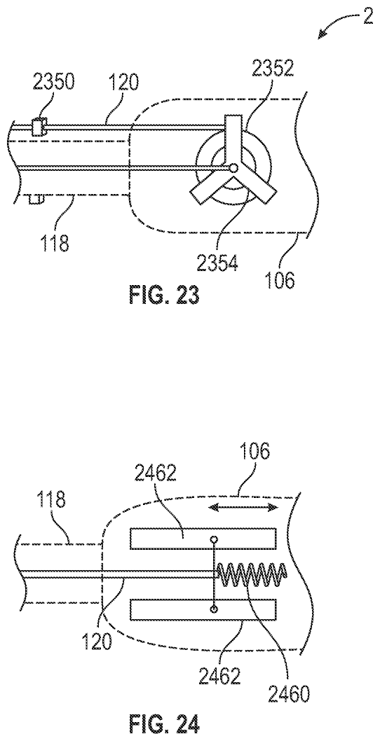

The housing 106 can include a container containing optional mechanical components, electrical or electronic components, or a combination thereof. The housing 106 can contain an anchor or mount structure to which the elongated shaft 118 can be attached. The housing 106 can provide an area in which the electrical interconnects 120 can be electrically coupled to the circuitry 116. The optional mechanical components can include the rotation wheel 112, an electrical interconnect management device (see, e.g., FIGS. 23-25) that can help keep the electrical interconnects 120 taut, or can aid in decoupling electrical ports 122, 124, a switch that, when activated or deactivated by the trigger 108, 110 can cause a device on a distal end of the shaft 118 (e.g., distal device 220, see FIG. 2) to move (e.g., such as to cut, cauterize, or the like), to turn on or off, or the like.

The trigger 108, 110, is a mechanical component that actuates another component or causes another component to move or otherwise change state. The trigger 108, 110 can be electrically or mechanically coupled to a device on the distal end of the shaft 118 (the distal device 220), such that upon pulling the trigger 108, 110 the distal device 220 actuates, such as changes state (e.g., closes jaws, advanced a blade, delivers an electrical signal, heats up, or the like). Upon releasing the trigger 108, 110 the distal device 220 can revert to the state it was in before the trigger 108, 110 was pulled.

The triggers 108 and 110 can control operation of different distal devices 220. For example, one trigger 108, 110 can cause an optical device to turn on, such as for illuminating a structure accessible through the second surgical device 104 and the other trigger 110, 108 can be used to perform an operation on the structure (e.g., a cauterize, cut, image or video capture, scrape, or the like).

The rotation wheel 112 can allow the user to rotate the shaft 118. Rotating the shaft 118 can, in turn, cause the distal device 220 to rotate. The distal device 220 can thus be rotationally oriented by adjusting the rotation wheel 112. By pushing the rotation wheel 112 away from the trigger 108, 110 and turning the rotation wheel 112, the shaft 118 (and the distal device 220) can be rotated clockwise or counterclockwise. By pushing the rotation wheel 112 towards the trigger 108, 110 and turning the rotation wheel 112, the shaft 118 (and the distal device 220) can be rotated the opposite direction.

The housing 114 can be configured for gripping by a user's hand. The housing 114 can be configured for a human hand to grip while allowing one or more fingers of the hand to extend to the trigger 108, 110 (in embodiments that include the trigger 108, 110). The housing 114, as illustrated, can include an integrally formed extension of the housing 106. In some embodiments, the housing 114 can be a separate component that can be attached to the housing 106.

The circuitry 116 can include one or more electrical or electronic components that can provide an electrical signal to the electrical port 122, receive an electrical signal from the electrical port 122, provide an electrical signal to the distal device, or a combination thereof. The electrical or electronic components can include one or more transistors, resistors; capacitors, diodes, inductors, power supplies (e.g., batteries), converters (e.g., power, current, voltage, analog to digital, digital to analog, or other converters), logic gates (e.g., AND, OR, XOR, negate, buffer, or the like), processing units (e.g., central processing units (CPUs), application specific integrated circuits (ASICs), field programmable gate arrays (FPGAs), graphics processing units (GPUs), or the like), memories (e.g., read only, programmable, random access, flash, a combination thereof, or the like), or the like.

A memory of the circuitry 116 can store data such as indicating a device identification, device type indication, or operating parameter indication, of the surgical device 102. The device identification or other data can be provided to the second device 104 (and ultimately a signal generator), through the electrical interconnects 120 and the electrical ports 122, 124. The device identification or other data can indicate to the signal generator a waveform (e.g., power level, current level, voltage level, frequency, amplitude, time between pulses, or the like) of an electrical signal provided to the device 102.

A button 117 can be electrically coupled to a switch of the circuitry 116. Upon activating the button 117 (“pushing the button”) the switch can complete an electrical circuit of the circuitry 116. The circuitry 116 can then cause electrical power to be actuated and provided to the distal device 220. The electrical power can cause the distal device 220 to heat up such as for a cauterizing, cutting, or burning operation.

The shaft 118 can include an elongated structure such as can be configured to fit through respective openings in the electrical ports 122, 124. The shaft 118 can include a lumen such as through which an electrical or mechanical or another component can travel. The electrical component can include one or more electrical interconnects, similar to or connected to the electrical interconnects 120. The mechanical component can include one or more wires, cords, or other elongated mechanical connection devices, such as connected between the trigger 108, 110 and the distal device.

The electrical interconnects 120 can provide an electrical path between the electrical port 122 and the circuitry 116 or the distal device 220. The electrical interconnects 120 can be electrically connected to one or more electrical connection features (e.g., 330, 332, 334, 336, 338, see FIG. 4) of the electrical port 122. The electrical interconnects 120 can include one or more individually insulated wires, a ribbon of insulated wires, a flexible circuit with meandering or other traces, or the like. A first electrical interconnect of the electrical interconnects 120 can be connected to provide a data signal from the circuitry 116 or the distal device 220 to the electrical port 124, through the electrical port 122. A second electrical interconnect of the electrical interconnects 120 can be connected to provide a power signal from the electrical port 122, through the electrical port 124, to the circuitry 116 or the distal device. The electrical interconnects 120 can be bendable, such as to allow them to extend or collapse as the device 102 is translated into or out of the openings 246, 346. The electrical interconnects 120 can retain the electrical port 122, such that the electrical port 122 remains around the shaft 118 when the electrical interconnects 120 are fully extended.

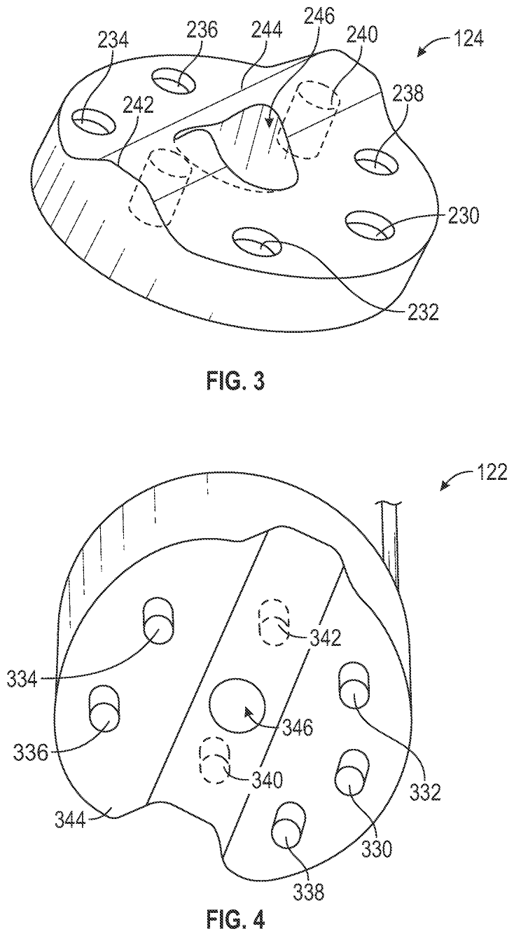

The electrical port 122 can provide one or more electrical signals from the electrical port 124 to the electrical interconnects 120 or vice versa. Embodiments of the electrical ports 124 and 122 are described elsewhere herein including FIGS. 3 and 4, among others.

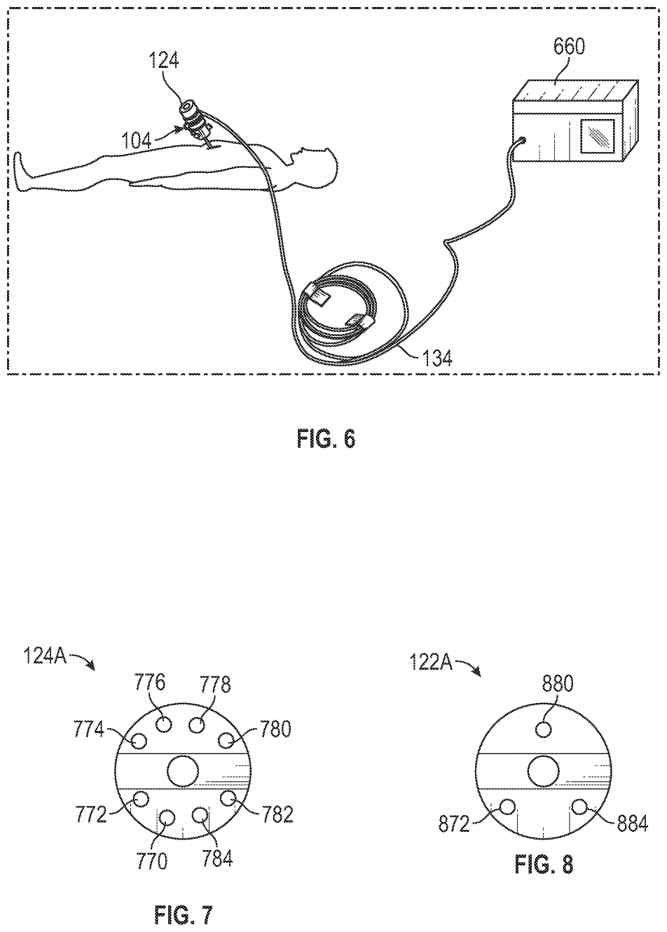

The second device 104 can include the electrical port 124, an electrical port support structure 126, an access stabilizer 128, and an electrical cord 134. The electrical port 124 can provide one or more electrical signals such as from an electrosurgical or other signal generator (see FIG. 6, among others) to the device 102, such as through the electrical port 122. Embodiments of the electrical port 124 are described elsewhere herein including FIG. 4, among others.

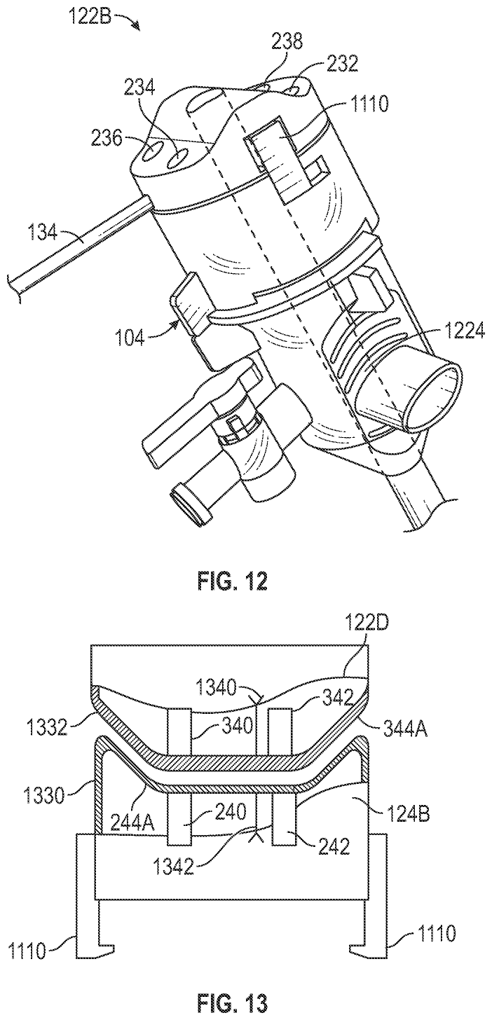

The support structure 126 can include a cannula (or other structure with a lumen) and a surrounding structure. The support structure 126 can be integrally formed with the electrical port 124 or the electrical port 124 can be configured to be mechanically coupled and decoupled from the support structure 126, such as by a user. The mechanical coupling can include mating a male or female connection feature on the support structure and a mating female or male connection feature of the electrical port 124. A mechanical decoupling can include detaching a male or female connection feature on the support structure from a mating female or male connection feature of the electrical port 124. The support structure 126 can include a lumen 1224 (see FIG. 12) overlapping (e.g., concentric or non-concentric) with an opening in the electrical port 124. More examples of details regarding the mechanical coupling and lumen 1224 is provided with respect to FIGS. 11, 12, and 27-30.

The access stabilizer 128 operates to hold open a natural orifice, incision, or other opening in the patient. The access stabilizer 128 can include a lumen overlapping with a lumen of the support structure 126 (the combined lumens are illustrated as the lumen 1224 in FIGS. 11 and 12), The lumen of the access stabilizer 128, when positioned to stabilize access, can extend into the patient. The lumen of the access stabilizer 128 can be configured to allow the shaft 118 to travel therethrough (longitudinally traverse through the lumen).

In the illustrated embodiment, the access stabilizer 128 can include a conical stabilizing device 130 and an elongated tubular structure 132 that can provide an external structure of the lumen. The stabilizing device 130 with a conical or other tapered shape can help prevent the access stabilizer 128 from falling through the opening in the patient. The widest part of the tapered shape can be larger than the opening, while the narrowest part of the tapered shape can be smaller than the opening. Such a configuration can allow the access stabilizer 128 to be partly in the patient and partly out of the patient while retaining access to the patient through the opening. The access stabilizer 128 illustrated in FIG. 1 is sometimes called a cannula.

The electrical cord 134 can include an insulated conductor that can provide one or more electrical signals such as between the electrical port 124 and the signal generator 606 (see FIG. 6, among others). The electrical cord 134 can include a male or female electrical connection feature 136 that can be plugged into the signal generator 606 to form an electrical path between the electrical port 124 and the signal generator 606.

FIG. 2 illustrates, by way of example, a diagram of an embodiment of the surgical device 102 with the shaft 118 removed from the opening in the electrical port 124. The surgical device 102 is similar to that illustrated in NG. 1, but with a distal device 220 visible on a distal end of the shaft 118. The distal device 220 can be attached to the distal end of the shaft 118. The distal device 220 can be retractable to within the shaft 118. The distal device 220 can be attached within the shaft 118, such that the distal device 220 is only partially exposed external to the shaft 118. The distal device 220 can include an optical device (e.g., a light emitting element, a camera, or the like), a mechanical device (e.g., forceps), electrical device, or a combination thereof (e.g., a cauterizer, cutter, gripper, suction, irrigation, or other like device). The distal device 220 can be controlled by the user, such as by using one or more of the triggers 108, 110, or the rotation wheel 112. The distal device 220 can be sized and shaped to fit through an opening 246 in the electrical port 124 and through the lumen 1224.

FIG. 3 illustrates, by way of example, a diagram of an embodiment of the electrical port 124. The electrical port 124 as illustrated can include male or female electrical or mechanical connection features 230, 232, 234, 236, 238. More or fewer electrical or mechanical connection features can be included, depending on the application. The connection features 230, 232, 234, 236, 238 as illustrated can include protrusions, sockets, or depressions or cutouts such as to receive mating female or male connection features or keying features of another electrical port 122 (see FIG. 4). The connection features 230, 232, 234, 236, 238 can be electrically connected to the electrosurgical or other signal generator, the circuitry 116, or the distal device.

The electrical port 124 as illustrated can include one or more keys or keyed features such as can provide orientation support. The keys illustrated in FIG. 3 include opposite polarity magnets 240, 242 and a contoured surface 244. The opposite polarity magnets 240, 242 can help guide or ensure that the connection between the connection features 230, 232, 234, 236, 238 and mating connection features of another electrical port (see FIG. 4) can only occur in a proper orientation. By including magnets that mate with (of opposite polarity to) the magnets 240, 242 in the other electrical port, any incorrect orientation of the electrical port will be repelled by the magnets 240, 242 and only the correct orientation between the electrical ports will be guided or allowed by the magnets 240, 242.

The contoured surface 244 can include one or more bumps, depressions, grooves, or the like such as can help reduce a number of orientations in which the electrical ports 200, 300 will mate. In the electrical port 124 of FIG. 3, the contoured surface 244 reduces the number of orientations in which the electrical ports 122, 124 can mate to two orientations. The magnets 240, 242, then reduce those two orientations to just one orientation. The magnets 240, 242 when mated with corresponding magnets 304, 342 of a complementary electrical port can provide magnetic force that holds the electrical ports 122, 124 together. In some embodiments, a hall effect sensor can be used in place of, or in addition to, the magnets 240, 242. The hall effect sensor measures a magnitude of a magnetic field. An output voltage of the hall effect sensor is directly proportional to the magnetic field strength through the sensor. The hall effect sensor can indicate whether there is a connection made between the electrical ports 122, 124.

FIG. 4 illustrates, by way of example, a diagram of an embodiment of the electrical port 122. The electrical port 122 can be configured to mate with the electrical port 124. The electrical port 122 as illustrated can include female or male electrical or mechanical connection features 330, 332, 334, 336, 338. More or fewer connection features can be included, depending on the application. The connection features 330, 332, 334, 336, 338 as illustrated can include one or more posts, pins, compression pins, bumps, or other features that extend from the contoured surface 344 of the mating connection features 230, 232, 234, 236, 238 of another electrical port (see FIG. 3). The connection features 330, 332, 334, 336, 338 can be electrically connected to the signal generator 606 (see FIG. 6), the circuitry 116, or the distal device 220.

The electrical port 122 as illustrated can include keys or keyed features that can help provide orientation support. The keys illustrated in FIG. 4 can include opposite polarity magnets 340, 342 and a contoured surface 344, The opposite polarity magnets 340, 342 can help guide or ensure that the connection between the connection features 330, 332, 334, 336, 338 and mating connection features 230, 232, 234, 236, 238 of another electrical port 124 (see FIG. 3) can only occur in one orientation. By including magnets that mate with (of opposite polarity to) the magnets 340, 342 in the other electrical port 122, any incorrect orientation of the electrical ports 122, 124 will be repelled by the magnets 240, 242, 340, 342 and only the correct orientation between the electrical ports 122, 124 will be guided or allowed by the magnets 240, 242, 340, 342,

FIG. 5 illustrates, by way of example, a perspective view diagram of a portion of an embodiment of the surgical device 102. The surgical device 102 in FIG. 5 can include a shroud 550 around the shaft 118 and the electrical interconnects 120. The shroud 550 can contain the electrical interconnects 120 within a confined space (within the shroud 550). This containment can help prevent unwanted catching on the wires or keep an area around the device 102 free of problematic wire clutter. The shroud 550 can be collapsible, rotatable, or a combination thereof. A rotatable shroud can help allow the electrical port 122 to freely orient with the electrical port 124. A collapsible shroud can help allow the user to push the shaft 118 (longitudinally translate the shaft 118) through the electrical ports 122, 124 and the surgical device 104 into the patient, while retaining the electrical interconnects 120 within the shroud 550. A length of the shroud 550 can be configured to provide an extension limit of the electrical port 122 from the housing 114. As the shaft 118 is removed from the openings 346, 246, the shroud 550 can become taut and cause the electrical and mechanical coupling between the electrical ports 122, 124 to break.

The shroud 550 can include or be made of a fabric, polymer, wire, fibers, or the like. A distal portion of the shroud 550 can be attached to the electrical port 122 outside of the electrical interconnects 120, such as to not interfere with an electrical connection between the electrical interconnects 120 and the electrical port 122. A proximal portion of the shroud 550 can be connected to the housing 106. The shroud 550 can be attached such that it does not contact the shaft 118. This can help allow the shaft 118 to move without affecting the shroud 550.

FIG. 6 illustrates, by way of example, a diagram of an embodiment of a medical system. The system as illustrated includes the surgical device 104 and a signal generator 660, electrically connected by the electrical interconnect 134. The electrical interconnect 134 can be connected, more specifically, between the electrical port 124 and the signal generator 660. The signal generator 660 can provide a power or data electrical signal to the electrical port 124, through the electrical interconnect 134. The power from the signal generator 660 can actuate or cause the distal device 220 (see FIG. 2) or the circuitry 116 (see FIG. 1) to operate, such as to provide light, capture image data, cauterize, burn, cut, irrigate, suction, or the like. The electrical signal from the signal generator 660 can cause a change in the circuitry 116 that configures operation of the distal device 220. In some embodiments, the signal generator 660 can receive a signal from the electrical port 124 (generated by the circuitry 116, through the electrical port 122 (see FIG. 1), or a combination thereof) that indicates data such as a device identification of the surgical device 102. The device identification can indicate to the signal generator 660 information such as device type or waveform parameters (e.g., frequency, amplitude, magnitude, power, pulse width, pulse duration, voltage or current level, or the like) of a signal to be provided to the circuitry 116 or the distal device 220.

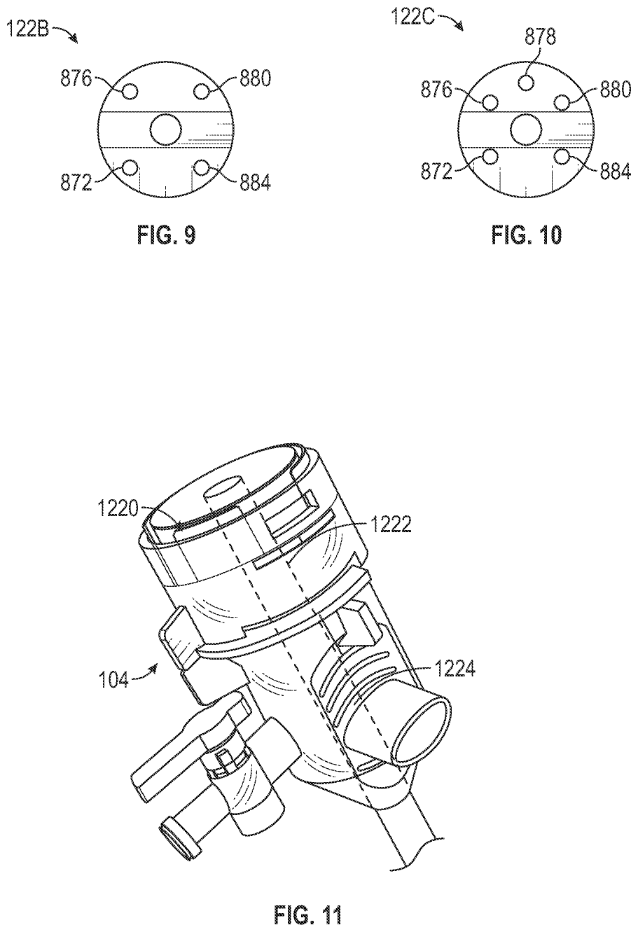

FIG. 7 illustrates, by way of example, a diagram of an embodiment of an electrical port 124A. Reference numbers with alphabetic suffixes indicate specific instances of the general component referred to without the suffix. Thus, the electrical port 124A is a specific instance of the electrical port 124. In some embodiments, the electrical port 124 can be configured to mate with electrical ports 122 with connection features of different configurations. This makes the electrical port 124 a sort of universal connector. The electrical port 124A is an example of such a universal electrical port 124A. In the embodiment of FIG. 7, the electrical port 124A can include a plurality of female or male electrical connection features 770, 772, 774, 776, 778, 780, 782, 784. The electrical port 124A can mate with electrical ports 122A (see FIG. 8), 122B (see FIG. 9), 122C (see FIG. 10) with different electrical configurations.

FIGS. 8, 9, and 10 illustrate, by way of example, diagrams of respective embodiments of the electrical port 122, specifically electrical port 122A, 122B, and 122C, respectively. The electrical connection features 770, 772, 774, 776, 778, 780, 782, 784 that form a circuit upon mating with electrical connection feature 872, 876, 878, 880, 884 can indicate a type of the surgical device 102. This can operate in addition to or in place of the device identification. In FIG. 8, electrical connections of the electrical port 122A will be made to the connection features 780, 784, 772 and this indicates that the surgical device 102 is different than a device that includes connection features made to the connection features 780, 776, 774, 772 as in the electrical port 122B of FIG. 9. The electrical connection features 770, 772, 774, 776, 778, 780, 782, 784 that form a circuit can thus indicate, to the signal generator 660 (see FIG. 6), the waveform parameters to provide to the surgical device 102.

FIG. 11 illustrates, by way of example, a diagram of an embodiment of the surgical device 104 in the form of a cannula. The surgical device 104 can include a cap 1220 such as that protects access to the internal structures thereof. The surgical device 104 can further include a female or male mechanical connection feature 1222 to which a male or female mechanical connection feature 1110 (see FIG. 12) of the electrical port 124 can be mated. The surgical device can further include a lumen 1224 extending from a proximal end thereof to a distal end thereof. The proximal end is the end to which the electrical port 124 can be mated. The distal end is the opposite end of the surgical device 104, the end configured to be located within the patient. The cap 1220 can be removed so that the electrical port 124 can be attached to the surgical device 104,

FIG. 12 illustrates, by way of example, a diagram of an embodiment of the electrical port 124 attached to the surgical device 104. The male or female mechanical connection feature 1110 of the electrical port 124 can be mated with a corresponding female or male connection feature 1222 of the surgical device 104 (occluded by the connection feature 1110 in FIG. 12, see FIG. 11). The connection feature 1110 can include a tab, decent, magnet, bump, threaded assembly, pin, ball or socket, or the like. The mating connection feature 1222 can include a corresponding feature such as that causes the electrical port 124 to be permanently or detachably connected to the surgical device 104. In some embodiments, the electrical port 124 can be integrally formed with the surgical device 104, such as to be part of the surgical device 104 and a separable component as shown in FIG. 12.

FIG. 13 illustrates, by way of example, a cross-section diagram of an embodiment of an electrical and mechanical mating between electrical ports 122D, 124B. Respective contoured surfaces 244A, 344A of the electrical ports 124B, 122D can include concave and mating convex shapes. The mating concave and convex shapes can cause the electrical port 122D to be centralized or aligned to the electrical port 124B. The keys or keyed features (e.g., in the form of magnets 240, 242, 340, 342 in the example of FIG. 13) can help guide proper orientation of the electrical ports 122D, 124B to each other. The keys can help properly rotationally align corresponding electrical connection features (see FIGS. 3, 4, among others) of the electrical ports 122D 124B.

In the example of FIG. 13, each of the electrical ports 122D, 124B can include a coating 1332, 1330 on an exposed surface. The coating 1332, 1330 can have a low coefficient of friction. The coefficient of friction of the coating 1330, 1332 can be less than the coefficient of friction of the contoured surface 244, 344. In some embodiments, the coefficient of friction can be less than 0.1, 0.05, some lower value, or some value therebetween. Reducing the coefficient of friction can help to allow the electrical ports 122D, 124B to properly orient to one another.

In the embodiment of FIG. 13, each of the electrical ports 122D, 124B can include an electrical connection feature such as in the form of respective communications circuitry 1340, 1342. The communications circuitry 1340, 1342 can provide a communication path that does not require physical contact. The communications circuitry 1340 can provide data or power wirelessly (e.g., near field, far field, inductively, capacitively, or the like) to the communications circuitry 1342 or vice versa. The communications circuitry 1340, 1342 can include electrical or electronic components (e.g., a transmit radio, receive radio, antenna, modulator, demodulator, amplifier, mixer, phase locked loop, a combination thereof, or other circuitry).

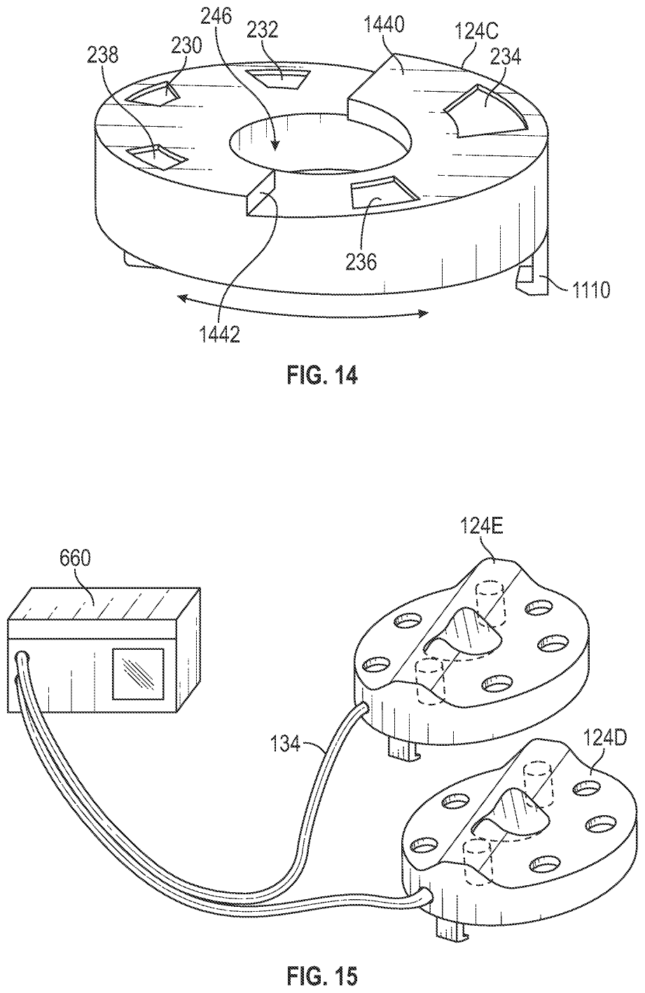

FIG. 14 illustrates, by way of example, a diagram of an embodiment of another embodiment of the electrical port 124C. The electrical port 124C provides an example of how the electrical ports 122, 124 can be oriented without requiring using magnets 240, 242, 340, 342. In the example of FIG. 14, the electrical port 124C can include electrical connection features 230, 232, 234, 236, 238 such as in the form of conductive pads. The electrical port 124C can include a key such as in the form of steps 1440, 1442. A mating electrical port 122 can be guided to the electrical port 124C, such as by inserting the shaft 118 of the surgical device 102 into the opening 246. The mating electrical port 122 can then twist such as until its mating steps contact the steps 1440, 1442.

So far, the discussion has emphasized a single electrical port 124 used in the surgical process. However, many surgical processes can include or benefit from using multiple access stabilizers or surgical devices 104. Each of the access stabilizers or surgical devices 104 can include a corresponding electrical port 124 attached thereto or integrally formed therewith.

FIG. 15 illustrates, by way of example, a diagram of an embodiment of multiple electrical ports 124D, 124E that can be electrically coupled in parallel with each other and coupled to the signal generator 660. The electrical ports 124D, 124E can include their own electrical interconnect coupled between the signal generator 660 and the electrical port 124D, 124E, respectively. The electrical interconnect 134 of the electrical ports 124D, 124E can be contained, at least partially, within the same housing or their corresponding housings are connected. The parallel electrical connection to the signal generator 660 need not include an electrical connection between the electrical ports 124D, 124E. In the parallel case, the electrical ports 124D, 124E can be electrically coupled through the signal generator 660 or the electrical interconnects 134 can be coupled to the signal generator 660.

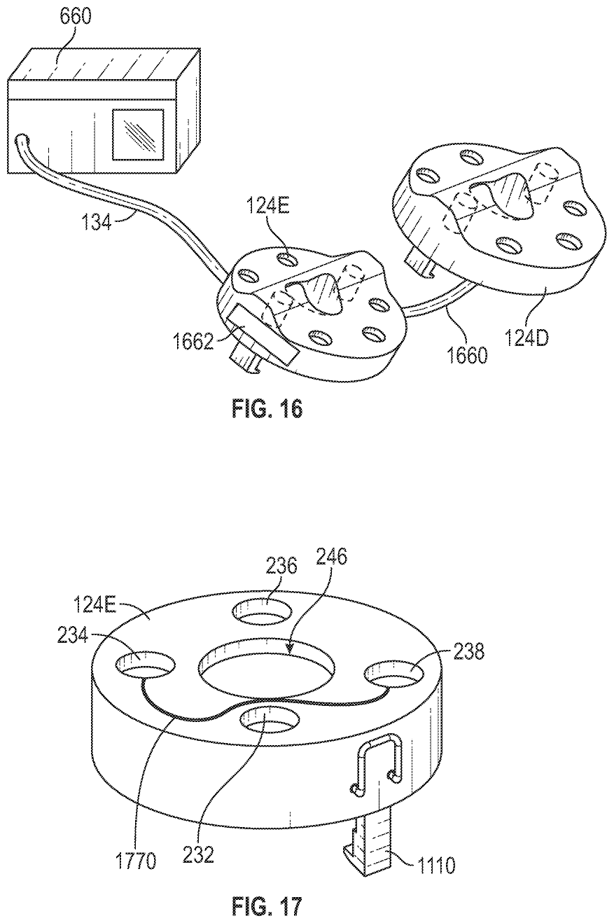

FIG. 16 illustrates, by way of example, a diagram of an embodiment of multiple electrical ports 124D, 124E such as can be electrically coupled in series to the signal generator 660. The series electrical connection to the signal generator 660 can include an electrical connection between the electrical ports 124D, 124E, such as via electrical interconnect 1660.

The signal generator 660 is optional. Electrical power can be provided without the signal generator 660. For example, one or more of the electrical ports 124C, 124D can include a battery 1662. The battery 1662 can provide temporary power to the circuitry 116 or the distal device 220. The battery 1662 can act as a backup or a replacement for the signal generator 660.

FIG. 17 illustrates, by way of example, a diagram of an embodiment of the electrical port 124E that can include multiple proper orientations with the electrical port 122. By electrically coupling opposing electrical connection features 234, 238 through an electrical interconnect 1770, the other opposing electrical connection features 232, 236 can provide electrical power to the device, A positive side of the electrical power can be provided through one of the electrical connection features 232, 236 and a negative side (e.g., ground, negative alternating current (AC), negative direct current (DC), or the like) can be provided through the other electrical connection feature 236, 232. The positive and negative power couplings to the surgical device 102 can be indiscriminate, so the electrical port 122 can properly couple in a first orientation and a second orientation 180 degrees from the first orientation, Other configurations can be used to provide flexibility in the number of orientations that provide proper electrical performance to the surgical device 102.

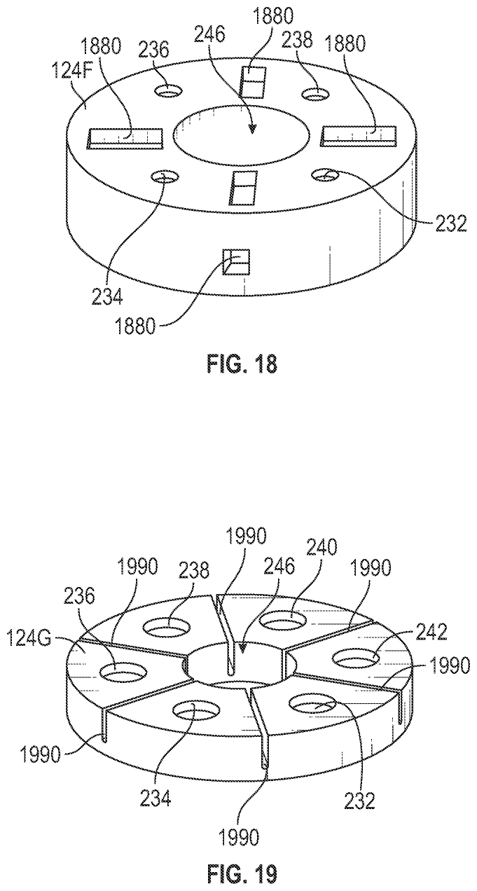

FIG. 18 illustrates, by way of example, a diagram of an embodiment of the electrical port 124F. The electrical port 124F as illustrated includes troughs 1880 situated between connection features 232, 234, 236, 238. The troughs 1880 can help prevent arcing between connection features 232, 234, 236, 238, The troughs 1880 can provide openings through which fluid can be provided internal to the patient (e.g., through the lumen 1224). The trough 1880 can include a hole in the side of the electrical port 124F and extend towards the opening 246. The trough 1880 can include a hole in the opening 246 through which fluid can be communicated to the lumen 1224.

FIG. 19 illustrates, by way of example, a diagram of yet another embodiment of the electrical port 124G. The electrical port 124G can include cutouts 1990 between connection features 232, 234, 236, 238. The width of the cutout 1990 can be sized to not interfere with alignment between the connection features 232, 234, 236, 238 and connection features of a mating electrical port 122, The cutout 1990 can extend from the side of the electrical port 124G to the opening 246. The cutout 1990 need not extend all the way to the side, the opening 246, or both.

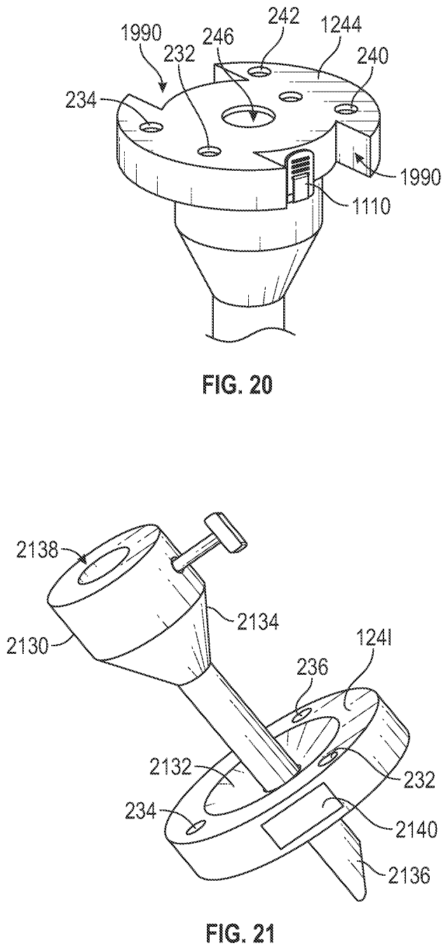

FIG. 20 illustrates, by way of example, an embodiment of the electrical port 124H. The electrical port 124H can include arcuate cutouts 1990 on opposing sides thereof. A mating electrical port 122 can include corresponding extensions that that at least partially fill the openings formed by the cutouts 1990.

So far, this description has emphasized embodiments of electrical ports 124 that can be coupled to a proximal end of a surgical device that includes an access stabilizer. The electrical port 124 can be situated on the patient and then the access stabilizer can be situated through the opening in the electrical port 124.

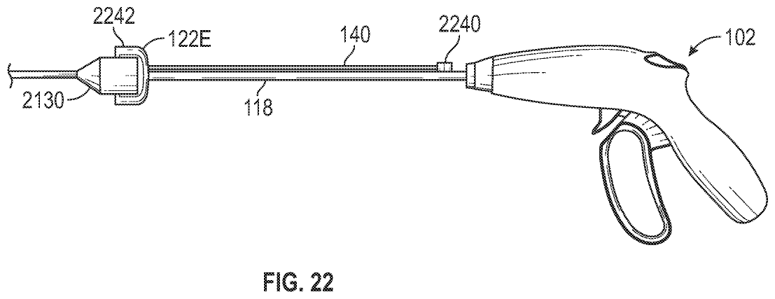

FIG. 21 illustrates, by way of example, a perspective view diagram of an embodiment of an electrical port 124I that can be situated more proximal than at least a portion of an access stabilizer 2130. FIG. 22 illustrates, by way of example, a diagram of an embodiment of a surgical device that can include an electrical port 122E such as can be configured to mate with the electrical port 124I or the electrical port 124H.

The electrical port 124I can be situated on the patient (e.g., on or in contact with the skin of the patient). An opening in the electrical port 124I (the opening through which the access stabilizer 2130 is shown as penetrating in FIG. 21) can be aligned with an orifice, incision, or other opening in the patient. The access stabilizer device 2130 can be situated through the opening and into the patient. A distal end of the access stabilizer device 2130 can be situated in the opening of the electrical port 124I and pushed into the opening in the patient. The access stabilizer 2130 can help keep the opening in the patient from collapsing, closing, or otherwise frustrating access to the insides of the patient.

The electrical port 124I as illustrated can include a contoured surface 2132 that can be frustoconical. The electrical port 124I can include an opening through which the access stabilizer 2130 can be situated. The contoured surface 2132 can be complementary to a convex contoured surface 2134 of the access stabilizer 2130. A lumen 2138 in the access stabilizer 2130 can extend from a proximal end through to a distal end of the access stabilizer 2130. The lumen 2138 can extend longitudinally through the access stabilizer 2130, such as to help provide access to the patient.

A mating electrical port 122E can be configured to include extended sidewalls 2242 or an extended male or female electrical connection feature that is configured to electrically connect with one or more of the electrical connection features 230, 232, 234 of the electrical port 124I. The mating electrical port 122E can include communications circuitry 2140 and can be wirelessly coupled to the electrical port 124I (see FIG. 13). The electrical port 124I can include communications circuitry 2140 (e.g., a transmit radio, receive radio, antenna, modulator, demodulator, amplifier, mixer, phase locked loop, or other communications circuitry). The communications circuitry 2140 can be wirelessly coupled to corresponding communications circuitry of the electrical port 122E. The wireless coupling between the electrical ports 122, 124 can include a near field, far field, inductive, or other wireless coupling.

FIGS. 23-25 illustrate, by way of example, respective embodiments of electrical interconnect management devices. In FIG. 23 the surgical device 102 can include a spool 2352 and coil spring 2354. The electrical interconnect 120 can be wound around the spool 2352. The coil spring 2354 can resist motion of the electrical interconnect 120 towards a distal end of the device 102. As the electrical interconnect 120 moves towards the distal end of the device 102 (away from the housing 106) tension in the coil spring 2354 increases. Upon the electrical port 122 being uncoupled from the electrical port 124, the coil spring 2354 causes the electrical interconnect 120 to be wound on the spool 2352. An optional stopper 2350 can prevent the electrical interconnect 120 from being completely internal to the housing 106. The stopper 2350 can also help protect the electrical connection between the electrical interconnect 120 and the electrical port 122, Without the stopper 2350 and upon retraction, the electrical port 122 can be retracted to the housing 106, which may cause stress on the electrical connection.

In the embodiment of FIG. 24, a spring 2462 can be coupled to rails 2462 situated in a track. The electrical interconnect 120 can be mechanically coupled to the rails 2462. The spring 2460 can resist motion of the electrical interconnect 120 away from the housing 106. Upon the electrical port 122 being uncoupled from the electrical port 124, the spring 2362 can cause the electrical interconnect 120 to be retracted towards the housing 106.

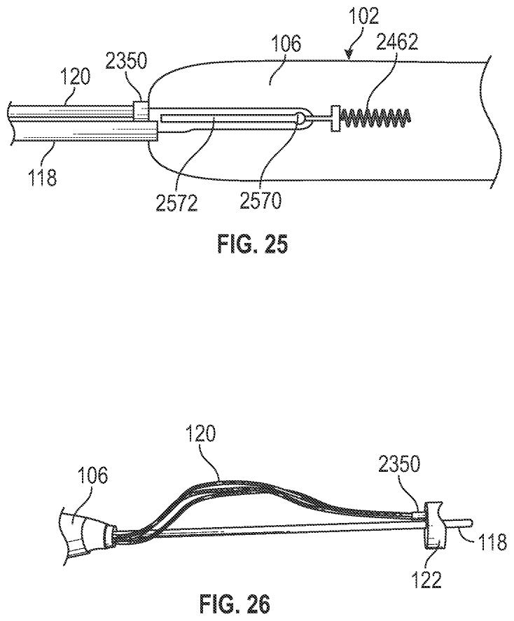

In the embodiment of FIG. 25, the spring 2462 can be coupled to a pulley 2570. The pulley 2570 can be situated on a track 2572 such as that allows the pulley to move longitudinally towards and away from the spring 2462. As the electrical port 122 is pulled further along the shaft 118, the tension in the spring 2462 increases and eventually overcomes the force of the mechanical coupling between the electrical ports 122, 124. The electrical ports 122, 124 then decouple and the electrical port 122 is retracted towards the housing 106.

FIG. 26 illustrates, by way of example, a diagram of an embodiment of the electrical interconnect 120 not wound around the shaft 118. Some embodiments discussed include the electrical interconnect 120 wound about the shaft 118. Such winding may cause a problematic inductance. The inductance may cause problems in communicating the electrical signal between the electrical ports 122, 124. Some embodiments, instead of including the electrical interconnect 120 travelling along the shaft 118 without winding around the shaft 118.

A length of the electrical interconnect 120 can be configured such that the electrical port 122 is free to travel along the shaft 118 without coming off the shaft 118. Thus, a maximum exposed length of the electrical interconnect 120 can be less than a length of the shaft 118.

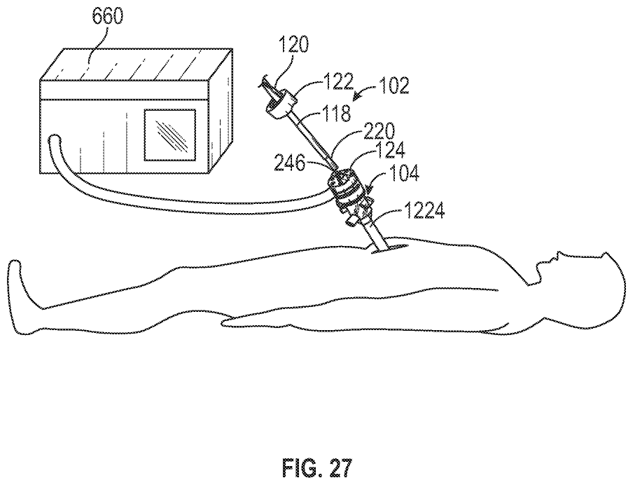

FIGS. 27, 28, and 29 illustrate, by way of example, perspective view diagrams of steps for electrically and mechanically coupling and decoupling the electrical ports 122, 124. In FIG. 27, electrical port 124 can be attached to the surgical device 104 before or after the surgical device 104 is situated to stabilize an opening in the patient. The electrical port 124 can be electrically connected to the signal generator 660 before or after the surgical device 104 is situated in the patient. In FIG. 27, the distal device 220 can be manipulated towards an opening 246 in the electrical port 124. The opening in the electrical port 124 can be concentric or overlapping with the lumen 1224, This can help allow the distal device 220 and the shaft 118 to be inserted into the patient through the opening 246 and the lumen 1224.

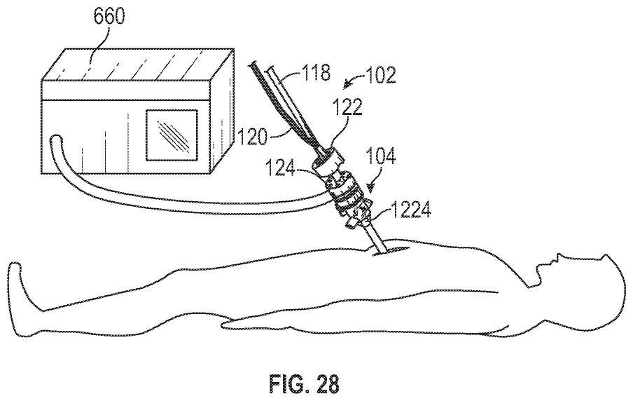

In FIG. 28, the shaft 118 of the device 102 can be longitudinally translated through openings in the electrical ports 122, 124, and into the lumen 1224. In performing the longitudinal translation, the electrical ports 122, 124 can be situated closer to each other than prior to the longitudinal translation (see FIG. 27).

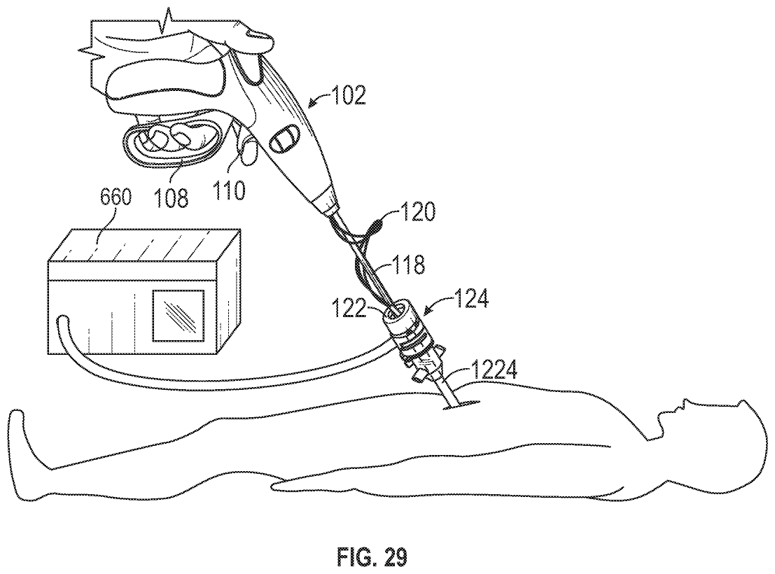

In FIG. 29, the shaft 118 can be situated further into the lumen 1224. This action can cause connections features of the electrical ports 122, 124 to mate. The mating forms an electrical and mechanical coupling between the electrical ports 122, 124. In the mated position, power or a signal from the signal generator 660 can be provided to the surgical device 102, through the surgical device 104. The user can then be free to operate the surgical device in the patient.

Then, when the user is finished or otherwise wishes to withdraw the surgical device 102 from the patient, the user longitudinally translates away from the surgical device 104. The electrical interconnects 120 will extend until a maximum length is reached. At the maximum length or when sufficient pull force is applied to the electrical port 124 (see FIGS. 23-25 for electrical interconnect management devices), the connection features of the electrical ports 124, 122 will disconnect, releasing the electrical ports 122, 124 from each other. FIG. 27 illustrates the surgical devices 102, 104 after the electrical ports 122, 124 are disconnected.

It is understood that the method steps disclosed herein can be performed in any order except as specified otherwise. Moreover, one or more of the following method steps can be combined with other steps; can be omitted or eliminated; can be repeated; and/or can separated into individual or additional steps.

The explanations and illustrations presented herein are intended to acquaint others skilled in the art with the invention, its principles, and its practical application. The above description is intended to be illustrative and not restrictive. Those skilled in the art may adapt and apply the invention in its numerous forms, as may be best suited to the requirements of a particular use.

Accordingly, the specific embodiments of the present invention as set forth are not intended as being exhaustive or limiting of the teachings. The scope of the teachings should, therefore, be determined not with reference to this description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventors did not consider such subject matter to be part of the disclosed inventive subject matter.

Plural elements or steps can be provided by a single integrated element or step. Alternatively, a single element or step might be divided into separate plural elements or steps.

The disclosure of “a” or “one” to describe an element or step is not intended to foreclose additional elements or steps.

While the terms first, second, third, etc., may be used herein to describe various elements, components, regions, layers and/or sections, these elements, components, regions, lavers and/or sections should not be limited by these terms. These terms may be used to distinguish one element, component; region, layer or section from another region; layer or section, Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings.

Spatially relative terms, such as “inner,” “outer,” “beneath,” “below,” “lower,” “above,” “upper.” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The disclosures of all articles and references, including patent applications and publications, are incorporated by reference for all purposes. Other combinations are also possible as will be gleaned from the following claims, which are also hereby incorporated by reference into this written description.

1-45. (canceled)

46. A surgical device comprising:
an elongated shaft configured to be guided via an access stabilizer;
a housing mechanically coupled to the shaft;
an electrical port at least partially around the shaft, the shaft extending through and able to longitudinally translate through an opening in the electrical port;
one or more electrical interconnects configured to receive an electrical signal from or provide the electrical signal to the electrical port; and
a shroud around the shaft, surrounding the electrical interconnects, and mechanically coupled to the housing.

47. The surgical device of claim 46, wherein the shroud is rotatable around the shaft.

48. The surgical device of claim 46, wherein the shroud is collapsible.

49. The surgical device of claim 48, wherein the electrical interconnects include electrical wires and the shroud completely surrounds a portion of the electrical wires outside of the housing.

50. The surgical device of claim 46, wherein the shroud includes fabric, polymer, wire, fiber, or a combination thereof.

51. The surgical device of claim 46, wherein the shroud is attached to the electrical port outside of the electrical interconnects.

52. The surgical device of claim 46, wherein the electrical port is configured to mate with a complementary electrical port mechanically coupled to or integrally formed with the access stabilizer.

53. The surgical device of claim 52, wherein the electrical port is configured to break an electrical and mechanical contact with the complementary electrical port during removal of the shaft from the opening.

54. The surgical device of claim 52, wherein the electrical port is keyed to the complementary electrical port.

55. The surgical device of claim 54, wherein the keying includes at least one of a magnet or a contoured surface configured to align the electrical port with the complementary electrical port.

56. The surgical device of claim 55, wherein the keying includes the contoured surface and the contoured surface includes one or more bumps, depressions, grooves, or a combination thereof.

57. The surgical device of claim 55, wherein the keying includes the contoured surface and the contoured surface reduces a number of orientations in which the electrical port and the complementary electrical port can mate.

58. The surgical device of claim 57, wherein the number of orientations in which the electrical port and the complementary electrical port can mate is two.

59. The surgical device of claim 58, wherein the keying further includes the magnet.

60. The surgical device of claim 59, wherein the magnet further reduces the number of orientations in which the electrical port and the complementary electrical port can mate to one.

61. A surgical device comprising:
an elongated shaft configured to be guided via an access stabilizer;
a housing mechanically coupled to the shaft;
an electrical port at least partially around the shaft, the shaft extending through and able to longitudinally translate through an opening in the electrical port, the electrical port is keyed to and configured to mate with a complementary electrical port mechanically coupled to or integrally formed with the access stabilizer; and
one or more electrical interconnects configured to receive an electrical signal from or provide the electrical signal to the electrical port; and

62. The surgical device of claim 61, wherein the electrical port is configured to break an electrical and mechanical contact with the complementary electrical port during removal of the shaft from the opening.

63. The surgical device of claim 61, wherein the keying includes at least one of a magnet or a contoured surface configured to align the electrical port with the complementary electrical port.

64. The surgical device of claim 61, wherein the keying includes the contoured surface and the contoured surface includes one or more bumps, depressions, grooves, or a combination thereof.

65. The surgical device of claim 61, further comprising a collapsible or rotatable shroud around the shaft, surrounding the electrical interconnects, and mechanically coupled to the housing.
